(19) United States
(12) Patent Application Publication
(10) Pub. No.: US 2020/0002681 A1
(43) Pub. Date: Jan. 2, 2020

(54) HOLLOW CELLULAR MICROFIBRE AND METHOD FOR PRODUCING SUCH A HOLLOW CELLULAR MICROFIBRE

(71) Applicants: UNIVERSITE DE BORDEAUX, BORDEAUX (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); INSTITUT D'OPTIQUE THEORIQUE ET APPLIQUEE, PALAISEAU CEDEX (FR)

(72) Inventors: LAETITIA ANDRIQUE, BORDEAUX (FR); GAËLLE RECHER, TALENCE (FR); KEVIN ALESSANDRI, BORDEAUX (FR); MAXIME FEYEUX, TALENCE (FR); PIERRE NASSOY, BORDEAUX (FR); ANDRÉAS BIKFALVI, GRADIGNAN (FR)

(21) Appl. No.: 16/491,662

(22) PCT Filed: Mar. 8, 2018
(86) PCT No.: PCT/FR2018/050541

§ 371 Date: Sep. 6, 2019

(30) Foreign Application Priority Data
Mar. 9, 2017 (FR) 1751941

(51) Int. Cl. C12N 5/071; C12N 5/077

(52) U.S. Cl. CPC C12N 5/0691; C12N 5/0697; C12N 5/0661; C12N 2537/10; C12N 2533/74; C12N 2506/45; C12N 2533/90

(57) ABSTRACT

The invention relates to a hollow cell microfibre comprising successively, organized around a lumen, at least one endothelial cell layer, at least one smooth muscle cell layer, an extracellular matrix layer, and optionally an outer hydrogel layer. The invention also relates to a process for fabricating such a hollow cell microfibre.

The invention relates to an artificial hollow cell microfibre having a structure, histology and mechanical properties similar to those of vessels in the animal vascular system. The invention also relates to a process for fabricating such a hollow cell microfibre. The invention has applications in particular in the field of tissue engineering and tissue grafts, to enable tissue vascularization, and in the pharmacological field, in particular for the study of candidate molecules with vascularization-related activity.

Recent years have seen the development of vascular tissue engineering with the aim of artificially recreating blood or lymphatic vessels, in particular to allow vascularization of tissues in vitro. For example, one method consists of moulding a cell-laden hydrogel around agarose-based tubes. The agarose tubes are then removed to create microtube networks (Bertassoni et al., Lab Chip. 2014 Jul. 7; 14(13):2202-2211). Another technique consists in pouring a collagen gel onto a gelatin or polydimethylsiloxane (PDMS) tube, which is removed once the collagen matrix has gelled (Backer et al., Lab Chip. 2013 Aug. 21; 13(16):3246-3252 and Jimenez-Torres et al., Methods Mol Biol. 2016; 1458:59-69). In all cases, the structure obtained is a block of agarose, collagen or other, in which the pseudovessels are formed. It is therefore not possible to extract them, to graft them and revascularize tissues. The use of these vessels is therefore limited to the in vitro study of anti-angiogenic, anti-thrombotic and other properties of molecules of interest. In addition, these solutions do not take into account the structure and histology of natural vessels, nor the constraints to which they are normally subjected.

Another approach consists in forming a tube by wrapping a layer of fibroblasts around itself before devitalizing said fibroblasts. Smooth muscle cells and endothelial cells are then cultured in the tube to reproduce cell microfibres mimicking blood vessels. However, the fabrication process for such microfibres is complex, requiring multiple operations and a development time of several months (Peck et al., Materials Today 14(5):218-224 May 2011).

Recently, microfibres containing endothelial cells covered by a layer of hydrogel have been obtained by coextrusion (Onoe et al., Nature Materials 31 Mar. 2013). However, these microfibres do not have mechanical properties comparable to those of blood or lymphatic vessels.

Thus, there remains a need for artificial hollow cell microfibres which can be individualized and handled and which have histology and mechanical properties similar to those of natural blood or lymphatic vessels.

### SUMMARY OF THE INVENTION

By working on novel ways of forming blood and lymphatic vessels, the inventors discovered that it is possible to fabricate hollow cell microfibres that histologically and mechanically reproduce vessels of the mammalian vascular system, such as blood vessels. More precisely, the inventors developed a process for encapsulating endothelial cells and smooth muscle cells in an alginate shell, within which the cells organize themselves into homocentric layers around a lumen. The process according to the invention makes it possible to obtain tubes of lengths and diameters that can be adjusted according to need. In particular, it is possible to produce tubes of a few centimetres and up to more than 1 metre. Similarly, the outer diameter of the tubes according to the invention can vary from 70 μm to more than 5 mm, so as to mimic all types of blood and lymphatic vessels, from veins to arteries. In addition, the lumen extends along the entire length of the tube, making the tubes perfusable. The vessels thus obtained can be easily individualized and handled.

A subject matter of the invention is therefore an artificial hollow cell microfibre comprising, successively, organized around a lumen
at least one endothelial cell layer;
at least one smooth muscle cell layer;
an extracellular matrix layer; and optionally
an outer hydrogel layer.

In a particular embodiment of the invention, the cell microfibre is a blood vessel or a lymphatic vessel.

Another subject matter of the invention is a process for preparing such a hollow cell microfibre, according to which a hydrogel solution and a cell solution comprising endothelial cells and smooth muscle cells in an extracellular matrix are concentrically coextruded in a crosslinking solution capable of crosslinking at least one polymer of the hydrogel solution.

### BRIEF DESCRIPTION OF THE FIGURES

FIG. 1: Cross-sectional representation of a hollow cell microfibre according to an exemplary embodiment of the invention, comprising successively, from the outside towards the inside, an outer alginate layer (1), an extracellular matrix layer (2), a smooth muscle cell layer (3), an endothelial cell layer (4) and a central lumen (5);

FIG. 2: Schematic representation of a concentric coextrusion system that can be used to produce cell microfibres according to the invention, wherein a first pump comprises an alginate solution (ALG), a second pump comprising an intermediate solution containing sorbitol (IS), and the third pump comprising a cell solution (C), these three solutions being brought to a coextrusion tip and the tip (6) being immersed in a crosslinking bath (7) to form the hollow cell microfibre (8);

FIG. 3: Microscopic views of the tubular structure of a cell microfibre obtained according to the process of the invention. Immediately after the formation of the tube (FIG. 3A), the cells are round and disposed inside the whole of the alginate tube; after 1 day of 3D culture (FIG. 3B), the cells anchor on the inner edges of the alginate tube, via the extracellular matrix, to form a lumen inside the tube;

FIG. 4: Study of the effect of the coextrusion rates of an alginate solution (a), a sorbitol solution (s) and a cell solution (c) on the thickness of the outer alginate layer in the obtained hollow cell microfibres;

FIG. 5: Study of the outer and inner diameters of different hollow cell microfibres obtained according to the process of the invention, as a function of the diameter of the coextrusion output nozzle (x-axis: 300 μm, 350 μm, 450 μm);

FIG. 6: View of an empty alginate tube with a diameter of 900 μm, obtained by extrusion with a 900 μm diameter outlet nozzle;

FIG. 7: Study of the contraction of the hollow cell microfibres according to the invention in the presence of endothelin 1 (ET1);

FIG. 8: Study of the increase in intracellular calcium concentration (I_{fluo}) in human umbilical vein endothelial cells (HUVEC) and in smooth muscle cells (SMC) of the hollow cell microfibres over time, under the effect of endothelin 1.

### DETAILED DESCRIPTION

Hollow Cell Microfibre

A subject matter of the invention is artificial hollow cell microfibres, the histology and mechanical and physiological properties of which mimic those of vessels in the animal vascular system, and in particular the mammalian vascular system.

The inventors have succeeded in producing in vitro microfibres based on smooth muscle cells and endothelial cells, the organization of which into concentric layers around a lumen makes said microfibres perfusable. In the context of the invention, “perfusable” means that it is possible to inject a fluid into said microfibre, within which it can circulate. Advantageously, the hollow cell microfibres according to the invention are also impermeable, in the sense that the fluid injected into said microfibres escapes little if at all through the thickness of the microfibres. The impermeability of a microfibre according to the invention depends mainly on the confluence of the cells in said microfibre. In particular, the confluence can be adapted by adjusting the number of cells injected during the formation of the microfibre. In addition, microfibres according to the invention can be handled, because they are individualized.

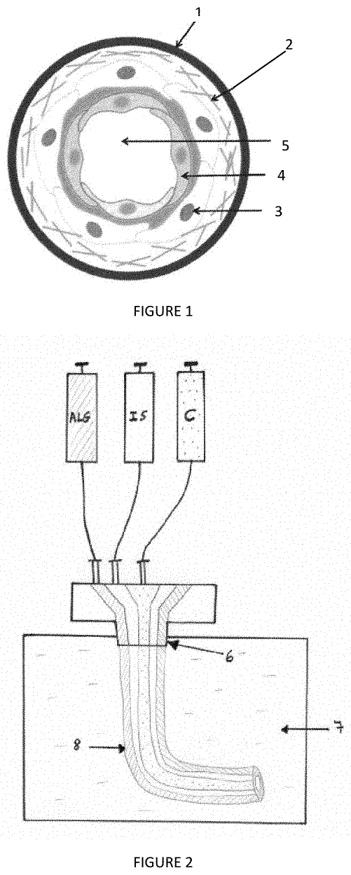

According to the invention, the cell microfibre is a hollow tubular structure, containing substantially homocentric layers, in the sense that they are successively organized around the same point. Thus, the central lumen 5 of the microfibre is bordered by the endothelial cell layer 4, which is surrounded by the smooth muscle cell layer 3, itself surrounded by an extracellular matrix layer 2 and optionally an outer hydrogel layer 1 (FIG. 1). A cross-section of the cell microfibre according to the invention thus comprises successive substantially concentric layers.

The lumen is generated, at the time the tube is formed, by smooth muscle and endothelial cells that self-assemble and spontaneously orient themselves with respect to the extracellular matrix layer. Advantageously, the lumen contains a liquid and more particularly culture medium.

In a particular embodiment of the invention, the hollow cell microfibre comprises an outer hydrogel layer. In the context of the invention, the “outer hydrogel layer” refers to a three-dimensional structure formed from a matrix of polymer chains swollen by a liquid, preferentially water. Advantageously, the one or more polymers in the outer hydrogel layer are polymers that can be crosslinked when subjected to a stimulus, such as temperature, pH, ions, etc. Advantageously, the hydrogel used is biocompatible, in the sense that it is not toxic to cells. In addition, the hydrogel layer must allow the diffusion of oxygen and nutrients to feed the cells contained in the microfibre and allow them to survive. The polymers in the hydrogel layer can be of natural or synthetic origin. For example, the outer hydrogel layer contains one or more polymers among sulfonate polymers, such as sodium polystyrene sulfonate, acrylate polymers, such as sodium polyacrylate, polyethylene glycol diacrylate, the compound gelatin methacrylate, polysaccharides, and in particular polysaccharides of bacterial origin, such as gellan gum, or of vegetable origin, such as pectin or alginate. In an embodiment, the outer hydrogel layer comprises at least alginate. Preferably, the outer hydrogel layer comprises only alginate. In the context of the invention, “alginate” refers to linear polysaccharides formed from β-D-mannuronate (M) and α-L-guluronate (G), salts and derivatives thereof. Advantageously, the alginate is a sodium alginate, composed of more than 80% G and less than 20% M, with an average molecular weight of 100 to 400 kDa (e.g., PRONOVA® SLG100) and a total concentration between 0.5% and 5% by density (weight/volume).

The outer hydrogel layer can increase the stiffness of the cell microfibre and thus facilitate its handling.

Advantageously, the hydrogel layer comprises cell-repellent polymers in order to facilitate, if necessary, the separation of said hydrogel layer from the cell microfibre or its degradation without affecting the structure of the cell microfibre.

In an embodiment of the invention, the cell microfibre has no outer hydrogel layer and comprises directly, as the outermost layer, an extracellular matrix layer.

Preferentially, the extracellular matrix layer forms a gel on the inner side of the hydrogel layer, i.e., the side facing the lumen of the microcompartment. The extracellular matrix layer consists of a mixture of proteins and extracellular compounds necessary for cell culture. Preferentially, the extracellular matrix comprises structural proteins, such as laminins containing the α1, α4 or α5 subunits, the β1 or β2 subunits, and the γ1 or γ3 subunits, vitronectin, laminins, collagen, as well as growth factors, such as TGF-beta and/or EGF. In an embodiment, the extracellular matrix layer consists of, or contains, Matrigel®, Geltrex®, collagen, and in particular collagen of type 1 to 19, optionally modified, gelatin, fibrin, hyaluronic acid, chitosan, or a mixture of at least two of these components.

According to the invention, the cell microfibre comprises smooth muscle cells, organized in one or more layers around and optionally at least partially in the extracellular matrix layer.

The smooth muscle cells can be selected from mammalian and particularly human vascular smooth muscle cells, lymphatic smooth muscle cells, digestive tract smooth muscle cells, bronchial smooth muscle cells, kidney smooth muscle cells, bladder smooth muscle cells, dermal smooth muscle cells, uterine smooth muscle cells and ciliary smooth muscle cells. Preferentially, the smooth muscle cells are selected from smooth muscle cells of lymphatic or vascular origin, such as umbilical artery smooth muscle cells, coronary artery smooth muscle cells, pulmonary artery smooth muscle cells, etc.

In a particular embodiment, the smooth muscle cells are smooth coronary artery muscle cells, such as human coronary artery smooth muscle cells.

In a particular embodiment, the smooth muscle cells are obtained from induced pluripotent stem cells, which have been forced to differentiate into smooth muscle cells.

According to the invention, the thickness of the one or more smooth muscle cell layers may vary according to the destination of the cell microfibre. “Thickness” means the dimension in a cross-section of the microfibre extending radially from the centre of that cross-section. The smooth muscle cells allow the microfibre to contract. It is therefore possible to adapt the contractile strength of the cell microfibre, depending on whether it is intended to be used as a blood vessel or a lymphatic vessel, but also according to the nature of said reproduced vessel (artery, vena cava, vein, venule, etc.). The skilled person knows the expected contractile force based on the vessel to be reproduced and thus knows how to adapt the thickness of the one or more smooth muscle layers, as well as the nature of the smooth muscle cells.

Advantageously, the one or more smooth muscle cell layers contain at least 95 vol %, preferentially at least 96%, 97%, 98%, 99% smooth muscle cells and matrix produced by said cells. The one or more smooth muscle cell layers may optionally comprise endothelial cells. Advantageously, the volume percentage of endothelial cells in the smooth muscle cell layer is less than 5%, preferably less than 4%, 3%, 2%, 1%.

According to the invention, the hollow cell microfibre comprises an endothelial cell layer, bordering and delimiting the central lumen.

The endothelial cells can be selected from mammalian and particularly human umbilical vein endothelial cells (UVEC), dermal microvascular endothelial cells (DMEC), dermal blood endothelial cells (DBEC), etc., dermal lymphatic endothelial cells (DLEC), coronary microvascular endothelial cells (CMEC), pulmonary microvascular endothelial cells (PMEC) and uterine microvascular endothelial cells (UtMEC).

In a particular embodiment, the endothelial cells are umbilical vein endothelial cells (UVEC), and in particular human umbilical vein endothelial cells (HUVEC).

In a particular embodiment, the endothelial cells are obtained from induced pluripotent stem cells, which have been forced to differentiate into endothelial cells.

Advantageously, the cell microfibre comprises a single layer of endothelial cells.

Advantageously, the one or more endothelial cell layers comprise at least 95 vol %, preferentially at least 96%, 97%, 98%, 99% endothelial cells and matrix produced by said cells. The one or more endothelial cell layers may optionally comprise smooth muscle cells. Advantageously, the volume percentage of smooth muscle cells in the endothelial cell layer is less than 5%, preferentially less than 4%, 3%, 2%, 1%.

According to the invention, it is possible, particularly according to the intended use of the hollow cell microfibre, to use animal cells of any origin, such as mouse cells, monkey cells, human cells, etc. Advantageously, the cells used to make the cell microfibre according to the invention are human cells.

In a particular embodiment, the average ratio of endothelial cells to smooth muscle cells, in cm², in a hollow cell microfibre of the invention is between 3:1 and 2:1

Advantageously, the inner diameter of the cell microfibre is between 50 μm and 500 μm, preferentially between 50 μm and 200 μm, more preferentially between 50 μm and 150 μm, even more preferentially between 50 μm and 100 μm, ±10 μm. The “inner diameter” refers to the diameter of the lumen of the microfibre. In a particular embodiment, the inner diameter of the cell microfibre is 100 μm. In another embodiment, the inner diameter is 70 μm.

The outer diameter of the cell microfibre can also vary. The “outer diameter” refers to the largest diameter of the microfibre. In the presence of an outer hydrogel layer, the outer diameter is advantageously between 250 μm and 5 mm. In the absence of an outer hydrogel layer, the outer diameter is advantageously between 70 μm and 5 mm, preferentially between 70 μm and 500 μm, more preferentially between 70 μm and 200 μm, even more preferentially between 70 μm and 150 μm, ±10 μm. In a particular embodiment, the outer diameter of the microfibre, in the presence of the outer hydrogel layer, is 300 μm. In a particular embodiment, the outer diameter of the microfibre, in the absence of the outer hydrogel layer, is 150 μm.

In a particular embodiment, the cell microfibre according to the invention comprises an outer hydrogel layer with a thickness of 100 to 150 μm, a cell thickness (endothelial cells and smooth muscle cells) of 150 to 200 μm and a lumen with a diameter of 100 to 150 μm.

Advantageously, the cell microfibre according to the invention has a length, or larger dimension, of at least 50 cm, preferentially at least 60 cm, 70 cm, 80 cm, 90 cm, 100 cm, 110 cm, or more.

Process for Preparing a Hollow Cell Microfibre

Another subject matter of the invention is a preparation process for obtaining a hollow cell microfibre according to the invention. More specifically, the invention proposes to encapsulate endothelial cells and smooth muscle cells in an outer hydrogel shell within which said cells will reorganize to form substantially concentric layers and provide a central lumen. Encapsulation is carried out by means of a concentric coextrusion process, in which the hydrogel solution is coextruded with the cell solution directly in a crosslinking bath, or crosslinking solution, comprising a crosslinking agent to crosslink the hydrogel and thus form the outer shell around the cells.

Any extrusion process allowing concentric coextrusion of the hydrogel and of the cells can be used. In particular, it is possible to produce cell microfibres according to the invention by adapting the method and the microfluidic device described in Alessandri et al., (PNAS, Sep. 10, 2013 vol. 110 no. 37 14843-14848; Lab on a Chip, 2016, vol. 16, no. 9, p. 1593-1604) or in Onoe et al. (Nat Material 2013, 12(6):584-90), so that all solutions are coextruded in a crosslinking bath, rather than above such a bath. For example, the process according to the invention is implemented by means of a double or triple concentric shell extrusion device as described in patent FR2986165.

In the context of the invention, “crosslinking solution” means a solution comprising at least one crosslinking agent adapted to crosslink a hydrogel comprising at least one hydrophilic polymer, such as alginate, when brought into contact with it. The crosslinking solution may be, for example, a solution comprising at least one divalent cation. The crosslinking solution may also be a solution comprising another known crosslinking agent of the alginate or the hydrophilic polymer to be crosslinked, or a solvent, for example water or an alcohol, adapted to allow crosslinking by irradiation or by any other technique known in the art.

Advantageously, the crosslinking solution is a solution comprising at least one divalent cation. Preferentially, the divalent cation is a cation used to crosslink alginate in solution. For example, it may be a divalent cation selected from the group consisting of Ca²⁺, Mg²⁺, Ba²⁺ and Sr²⁺, or a mixture of at least two of these divalent cations. The divalent cation, for example Ca²⁺, can be combined with a counterion to form for example CaCl₂ or CaCO₃ solutions, well known to the skilled person. The crosslinking solution may also be a solution comprising CaCO₃ coupled to glucono-delta-lactone (GDL) forming a CaCO₃-GDL solution. The crosslinking solution may also be a mixture of CaCO₃—CaSO₄-GDL.

In a particular embodiment of the process according to the invention, the crosslinking solution is a solution comprising calcium, in particular in the Ca²⁺ form.

The skilled person is able to adjust the nature of the divalent cation and/or the counterion, as well as its concentration, to the other parameters of the process of the present invention, in particular to the nature of the polymer used and to the desired rate and/or degree of crosslinking. For example, the concentration of divalent cation in the crosslinking solution is between 10 and 1000 mM.

The crosslinking solution may include components, well known to the skilled person, other than those described above, to improve the crosslinking of the hydrogel sheath under specific conditions, particularly time and/or temperature.

Advantageously, the endothelial cells were first cultured in a culture medium containing vascular endothelial growth factors (VEGF) to promote endothelial formation and angiogenesis. In an exemplary embodiment, the endothelial cells were first cultured in the medium EGM-2®.

Advantageously, the smooth muscle cells were first cultured in a culture medium containing growth factors adapted to the culture of smooth muscle cells, such as transforming growth factor β1, EGF, bFGF, etc. In an exemplary embodiment, the smooth muscle cells were first cultured in SmGM2® (Lonza) or in a culture medium specifically adapted to smooth muscle cells marketed by PromoCell (e.g., HCASMC®, HAoSMC® medium, etc.).

The cell solution used for coextrusion comprises endothelial cells and smooth muscle cells suspended in the extracellular matrix.

In a particular embodiment, the cell solution comprises between 20 and 30 vol % cells and between 70 and 80 vol % extracellular matrix.

The volume ratio of endothelial cells to smooth muscle cells in the cell solution is advantageously between 3:1 and 2:1.

According to the process of the invention, coextrusion is carried out in such a way that the hydrogel solution surrounds the cell solution.

In a particular embodiment, coextrusion also involves an intermediate solution, comprising sorbitol. In this case, coextrusion is carried out in such a way that the intermediate solution is disposed between the hydrogel solution and the cell solution (FIG. 2A).

In a particular embodiment, the extrusion rate of the alginate solution is between 1 and 10 ml/h, preferentially between 2 and 5 ml/h, even more preferentially equal to 3 ml/h and preferably equal to 2 ml/h, ±0.5 ml/h.

In a particular embodiment, the extrusion rate of the intermediate solution is between 0.1 and 5 ml/h, preferentially between 0.5 and 1 ml/h, even more preferentially equal to 0.5 ml/h, ±0.05 ml/h.

In a particular embodiment, the extrusion rate of the cell solution is between 0.1 and 5 ml/h, preferentially between 0.5 and 1 ml/h, even more preferentially equal to 0.5 ml/h, ±0.05 ml/h.

The coextrusion rate of the different solutions can be easily adjusted by the skilled person, in order to adapt the inner diameter of the microfibre and the thickness of the hydrogel layer.

In all cases, the extrusion rate of the hydrogel solution is higher than the extrusion rate of the cell solution and optionally of the intermediate solution. In particular, the extrusion rate of the hydrogel solution is at least two, three or four times faster than the extrusion rate of the cell solution.

Preferentially, the extrusion rates of the cell solution and of the intermediate solution are identical.

In a particular embodiment of the process according to the invention, the extrusion rate of the hydrogel solution is 2 ml/h, ±0.05 ml/h, and the extrusion rate of the cell solution and of the intermediate solution is 0.5 ml/h, ±0.05 ml/h.

In another particular embodiment of the process according to the invention, the extrusion rate of the hydrogel solution is 9 ml/h, ±0.05 ml/h, and the extrusion rate of the cell solution and of the intermediate solution is 3 ml/h, ±0.05 ml/h.

In another particular embodiment of the process according to the invention, the extrusion rate of the hydrogel solution is 3 ml/h, ±0.05 ml/h, the extrusion rate of the cell solution is 2 ml/h, ±0.05 ml/h, and the extrusion rate of the intermediate solution is 1 ml/h, ±0.05 ml/h.

In another particular embodiment of the process according to the invention, the extrusion rate of the hydrogel solution is 2 ml/h, ±0.05 ml/h, and the coextrusion rate of the cell solution and of the intermediate solution is 0.5 ml/h, ±0.05 ml/h.

In another particular embodiment of the process according to the invention, the extrusion rate of the hydrogel solution is 2 ml/h, and the coextrusion rate of the cell solution and of the intermediate solution is 1 ml/h, In another particular embodiment of the process according to the invention, the extrusion rate of the hydrogel solution is 2 ml/h, ±0.05 ml/h, the extrusion rate of the cell solution is 0.5 ml/h, ±0.05 ml/h, and the extrusion rate of the intermediate solution is 1.5 ml/h, ±0.05 ml/h.

In another particular embodiment of the process according to the invention, the extrusion rate of the hydrogel solution is 2 ml/h, ±0.05 ml/h, the extrusion rate of the cell solution is 1.5 ml/h, ±0.05 ml/h, and the extrusion rate of the intermediate solution is 0.5 ml/h, ±0.05 ml/h.

In a particular embodiment of the process according to the invention, as shown in FIGS. 2A and 2B, the crosslinking solution, the intermediate solution and the cell solution are loaded into three concentric compartments of a coextrusion device, so that the crosslinking solution (ALG), forming the first flow, surrounds the intermediate solution (IS) which forms the second flow, which itself surrounds the cell solution (C) which forms the third flow. The tip 6 of the extrusion device, through which the three flows exit, opens into the crosslinking solution 7, so that at the exit of the tip 6 a tube 8 is formed. The first flow is the rigid outer hydrogel shell. The second flow is the intermediate shell and the third flow is the internal shell containing the cells.

The process according to the invention allows smooth muscle cells and endothelial cells to be encapsulated in an outer hydrogel sheath. Surprisingly, the inventors observed that after only a few hours, the cells contained in this hydrogel sheath reorganize themselves, such that the endothelial cells delimit an internal longitudinal lumen extending over the entire length of the cell microfibre, and that the smooth muscle cells orient themselves outwardly with respect to the lumen. The presence of extracellular matrix during coextrusion seems necessary for the cells to anchor themselves to the matrix and thus spread, divide and proliferate. The matrix also reduces the risk of apoptosis of the cells inside the cell microfibre and promotes cell reorganization within the hydrogel sheath.

Advantageously, the cell microfibre obtained by coextrusion is maintained in a suitable culture medium for at least 10, preferentially at least 20 h, even more preferentially at least 24 h before being used. This latency time advantageously allows the cells to reorganize themselves in the hydrogel sheath to form concentric layers around a lumen, as described above.

According to the invention, it is possible to directly use the hollow cell microfibre obtained by coextrusion, i.e., a microfibre comprising a hydrogel sheath, or to proceed to hydrolysis of said sheath in order to recover a hydrogel-free microfibre.

Applications

The hollow cell microfibres forming the subject matter of the present invention can be used for many applications, in particular for medical or pharmacological purposes.

The cell microfibres according to the invention can be used in particular for tests to identify and/or validate candidate molecules having an action on all or part of the vascular system, and in particular on blood or lymphatic vessels. For example, such microfibres can be used to test the anti-angiogenic, anti-thrombotic, blood pressure regulating, blood gas transport regulating, etc., properties of candidate molecules.

The hollow cell microfibres according to the invention can also be used in tissue engineering to vascularize synthetic biological tissue samples and thus increase their viability. Such vascularized tissue samples can be used, for example, by the pharmaceutical and cosmetic industries to perform in vitro tests, particularly as an alternative to animal testing.

Similarly, the hollow cell microfibres according to the invention can be used in regenerative medicine to allow vascularization of synthetic organs, such as skin, cornea, liver, etc., tissues obtained by 3D printing or other means, before grafting them into a subject.

### EXAMPLES

### Example 1: Protocol for Obtaining a Hollow Cell Microfibre

Material & Method

Cells:

Human umbilical vein endothelial cells (HUVEC) cultured in a culture medium comprising VEGF in passage 3 (P3), 4 (P4) or 5 (P5), provided cryopreserved in liquid nitrogen at −80° C. (PromoCell®, item c-12205).

Human coronary artery smooth muscle cells, in passage 2 (P2), provided cryopreserved in liquid nitrogen at −80° C. (Lonza, item CC-2583).

Media:

Endothelial cell culture medium: PromoCell EGM2® Kit (item C-22111) (medium at +4° C. and supplements at −20° C.).

Endothelial cell detachment media: Detach KIT® [Hepes BSS (30 mM HEPES)+Trypsin/EDTA Solution (0.04%/0.03%)+Trypsin Neutralizing Solution (TNS)] (PromoCell, item C-41210).

Endothelial cell freezing medium: Cryo-SFM (PromoCell, item C-29912).

Smooth muscle cell culture medium: SmGm2-Bulletkit® (Lonza, item CC-3182) (medium at +4° C. and supplements at −20° C.).

Smooth muscle cell detachment medium: Detach KIT® (PromoCell, item C-41210).

Smooth muscle cell freezing medium: Cryo-SFM (PromoCell, item C-29912).

Solutions:

Crosslinking solution: 100 mM CaCl₂

Intermediate solution: 300 mM sorbitol

Hydrogel solution: 2.5% w/v alginate (LF200FTS) in 0.5 mM SDS

Extracellular matrix: Classic Matrigel® (without phenol red and with growth factors)

Treatment of Endothelial Cells (HUVEC):

Amplification

P3 HUVEC are thawed and amplified according to standard protocols up to P5, P6 or P7, coextrusion being carried out with cells between P5 and P7.

Treatment of Smooth Muscle Cells (SMC):

Amplification

P2 SMC cells are thawed and then cultured according to standard protocols up to P5, P6 or P7, coextrusion being carried out with cells between P5 and P7.

Coextrusion System
Three sterile Hamilton 12 ml syringes, one containing 2.5% alginate and the other two containing 300 mM sorbitol
Strandard Teflon tubing, diameter 13
neMESYS® syringe pump (CETONI) and associated software
3D printed injection chip (see publication Alessandri K et al., 2016)

Extrusion Process
Take up 30 μl of cells (½ SMC and ½ HUVEC) in 60 μl of Matrigel®.
coextrude the three solutions according to the method described in Alessandri et al. 2016 (FIG. 2A) with extrusion rates of 2 ml/hour for alginate and 0.5 ml/hour for sorbitol solution and cell solution, maintaining the tip of the extrusion device immersed in the crosslinking solution (FIG. 2B).

Results

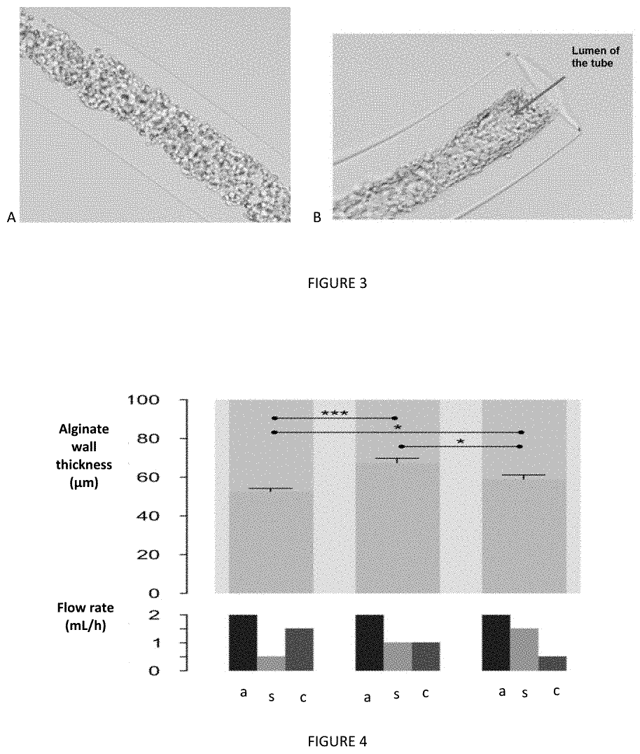

Coextrusion of the three solutions in a Ca²⁺ solution as described above produced tubes, or hollow cell microfibres, approximately 1 metre long and with an outside diameter of 300 μm. After 24 h (FIG. 3B), the cells reorganized and self-assembled inside the alginate tube so as to create a central lumen with a diameter of about 150 μm. The tube then successively comprises, and organized concentrically around the lumen, a HUVEC layer, a SMC layer, a Matrigel® layer and a crosslinked alginate layer.

### Example 2: Characterization of the Hollow Cell Microfibres

The hollow cell microfibres obtained in Example 1 were characterized using specific markers by immunofluorescence and confocal microscopy. Cell reorganization within the alginate shell was monitored by video microscopy.

Material & Method

Immunolabeling:

The cell microfibres, or tubes, were fixed at different times (D1/D5), with 4% paraformaldehyde diluted in DMEM without phenol red (PAN), overnight at 4° C.

The cells of the tubes were then permeabilized (30 min in 1% Triton in DMEM without phenol red, at room temperature with shaking). The nonspecific sites of the cells were saturated for one hour at 4° C. in a 1% bovine serum albumin (BSA)/2% foetal calf serum (FCS) solution.

The cell microfibres were then exposed to specific primary antibodies, each directed against a protein of interest:
CD31: specific marker of the endothelial cell membrane
aSMA (alpha smooth muscle actin): specific marker of the SMC cytoskeleton
VE-cadherin: specific marker of endothelial cell junctions and of formation of an impermeable endothelium
tubulin: specific marker of thecytoskeleton
KI67: specific marker of cell proliferation
aCaspase3: specific marker of apoptosis.

The primary antibody was diluted 1/100 in DMEM without phenol red+1% BSA/2% FCS overnight with shaking at 4° C. After 2×15 min of washing in DMEM without phenol red, the tubes were incubated with a secondary antibody (which will specifically recognize the primary antibody) coupled to a fluorochrome, diluted 1/1000 in DMEM without phenol red+1% BSA/2% FCS for 1 h at room temperature. After 2×15 min of washing in DMEM without phenol red, the tubes were analysed by confocal microscopy to visualize the fluorescence.

Results:
D1: 1 day after the formation of the tube, the cells are organized as follows: SMC (specific marker aSMA, alpha smooth muscle actin) on the Matrigel® side and HUVEC (specific marker CD31) on the lumen side. Both cell types proliferate (marker KI67 positive) and have very little cell death (little specific caspase 3 staining).
D5: 5 days after formation of the tube, the cell junctions become tight: the HUVEC contour is much more visible with cells closer and closer together. This phenomenon corresponds to “endothelialization”, i.e., the formation of an endothelium whose function is to become impermeable. In addition, at D5, the cells stop proliferating (loss of the KI67 signal) but do not die (no increase in the caspase 3 signal), indicating that the cells are entering quiescence, as is the case in a normal human vascular endothelium.

### Example 3: Evaluation of the Perfusion Capacity of Hollow Cell Microfibres

The perfusability of the microfibres was also assessed by connecting them to an injection system comprising fluorescent solutions.

A system for perfusing hollow cell microfibres was developed using glass Pasteur pipettes pulled under flame to a diameter corresponding to the inner diameter of the cell microfibres, i.e., 150 μm. The pulled pipettes were connected to a syringe containing culture medium (PromoCell EGM2®), itself connected to a syringe pump to allow fluid perfusion at a physiological rate of 50 μL/min. The rate of perfusion may vary according to the inner diameter of the cell microfibre.

The cell microfibres are cut into pieces a few centimetres long and placed in culture medium in a 3 cm Petri dish under a binocular magnifying glass. They are then connected to the tip of the pulled Pasteur pipettes.

The complete system (cell microfibre/culture medium, pulled pipette, syringe) is then re-cultured (incubator at 37° C., 5% CO₂) and allows the perfusion of EGM2® permanently into the vascular tubes.

An identical perfusion system was used to check the impermeability of the cell microfibres. Fluorescent tracer (200 μL) was injected into the cell microfibres according to the invention (HUVEC+SMC), as well as into cell microfibres containing only endothelial cells and into an alginate tube (500 kDa or 20 kDa fluorescein isothiocyanate (FITC)-dextran, Sigma-Aldrich) at a physiological rate of 50 μl/min.

The rate of diffusion of each fluorescent tracer through the alginate was filmed and quantified.

Results
Negative control (cell-free alginate tube+500 kDa FITC-dextran): The high molecular weight dextran molecules do not pass through the pores of the alginate;
Positive control (cell-free alginate tube+20 kDa FITC-dextran): The low molecular weight dextran molecules easily diffuse through the pores of the alginate;
Alginate/HUVEC/SMC microfibre according to the invention+20 kDa FITC-dextran: the low molecular weight dextran molecules diffuse little if at all through the cell layers, which make the microfibre impermeable;
HUVEC/SMC microfibre according to the invention (after hydrolysis of the outer alginate layer)+20 kDa FITC-dextran: the diffusion rate of the dextran molecules through the cell layers is close to that observed for the microfibre according to the invention still comprising the outer alginate layer.

Thus, even in the absence of the outer alginate layer, the structure, the perfusability and the impermeability of the hollow cell microfibre according to the invention of the tube are maintained.

### Example 4: Controlled Modification of the Thickness of the Outer Alginate Layer

Three hollow cell microfibres were fabricated, according to the protocol described in Example 1, by varying the extrusion rates of the sorbitol solution and of the cell solution for a constant alginate extrusion rate. The extrusion rates for the 3 hollow cell microfibres are summarized in the table below.

Extrusion Rates of the Different Solutions

| | Alginate | Sorbitol | Cell suspension |
|---|---|---|---|
| Microfibre 1 | 2 ml/h | 1 ml/h | 1 ml/h |
| Microfibre 2 | 2 ml/h | 0.5 ml/h | 1.5 ml/h |
| Microfibre 3 | 2 ml/h | 1.5 ml/h | 0.5 ml/h |

The purpose of this experiment is to verify 1/the reproducibility of dimensions of the hollow cell microfibres with identical parameters, 2/the impact of flow rates on the thickness of the outer alginate wall.

Results

When the two inner flows (sorbitol solution and cell solution) are extruded at the same rate, the outer alginate layer of the resulting microfibres is thicker (FIG. 4). Even with a constant ratio (alginate flow rate)/[(sorbitol flow rate)+(cell suspension flow rate)], the kinetic asymmetry of the flows of sorbitol and of the cell suspension leads to the production of a thinner outer alginate layer, with a more pronounced effect when the sorbitol flow rate is the lowest.

These experiments confirm that the outer and inner diameters of the hollow cell microfibres can be adjusted by the coextrusion system. In particular, the results show that it is possible to slightly, but significantly, vary the thickness of the outer alginate layer by varying the flow rate of the coextruded solutions.

### Example 5: Controlled Modification of the Diameter of the Hollow Cell Microfibres

Hollow cell microfibres were fabricated, according to the protocol described in Example 1, by modifying the outlet nozzle of the concentric coextrusion system solutions (see coextrusion nozzle/tip 6, FIG. 2), to obtain an output nozzle with a diameter of 300 μm, 350 μm, 450 μm and 900 μm. With the 900 μm nozzle, the alginate solution was extruded alone to produce empty alginate tubes (without cell suspension).

The outer diameter and the inner diameter, i.e., the lumen of the microfibres, were measured after synthesis of said microfibres.

Results

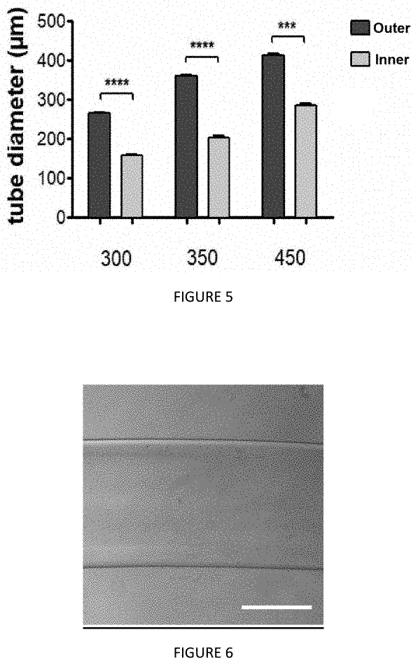

The results presented in the table below and in FIG. 5 confirm that it is possible to modify the dimensions of the microfibres and modifying the diameter of the coextrusion tip of the coextrusion system. In addition, the obtaining of a hollow alginate tube with a diameter of 900 μm with a 900 μm outlet nozzle (FIG. 6) confirms that the process according to the invention makes it possible to obtain perfectly controlled hollow cell microfibres with a perfectly controlled diameter.

Outer and Inner Diameters of the Microfibres as a Function of Output Nozzle Diameter

| Nozzle diameter | 300 | μm | 350 | μm | 450 | μm |
|---|---|---|---|---|---|---|
| Outer diameter | 264.68 μm ± 10.9 μm | | 360.37 ± 10.8 | μm | 448.53 μm ± 12.33 μm | |
| Inner diameter | 158.17 ± 6.32 | μm | 203.11 ± 16.53 | μm | 321.62 ± 21.47 | μm |

### Example 6: Measurement of the Contractility of the Hollow Cell Microfibres and of Calcium Fluxes

Material & Method

Hollow cell microfibres with an inner diameter of about 400 μm were produced according to Example 1.

After 24 h of culture, the microfibres are incubated for 45 min in the presence of a calcium-sensitive fluorescent probe, Fluo-4 AM (ThermoFisher scientific, F23917, 50 μg dissolved in 4 μL pluronic acid −20% in DMSO-, then diluted in 800 μL of EGM2, final concentration: 50 μM), at 37° C. The AM (acetoxymethyl) group allows the molecule to cross the plasma membrane, it is cleaved by intracellular esterases, which traps the probe in the cytoplasmic compartment. Variations in fluorescence signal intensity provide information on qualitative variations (non-ratiometric probe) of free calcium available at the binding site of the molecule. This information is an indirect measure of the activation of signalling pathways involving extracellular calcium entry, and/or release of calcium reserves from the endoplasmic reticulum.

After rinsing in culture medium EGM2, the microfibres are imaged in epifluorescence with a stereoeomicroscope. A vasoconstrictor specific to blood vessels, endothelin 1 (ET1, 0.1 μM), is applied in the vicinity of the tube, in the culture medium. The fluorescence signal is collected before, during and after application of the vasoconstrictor.

The collected data make it possible to measure: 1/the contraction of the microfibres (measurement of the outer diameter), 2/the variations in intensity of the fluorescence signal of Fluo-4 AM, (intracellular calcium is the second messenger involved in the signalling cascade triggering the contraction of muscle fibres and therefore the decrease in the inner diameter of the vesseloid).

Results

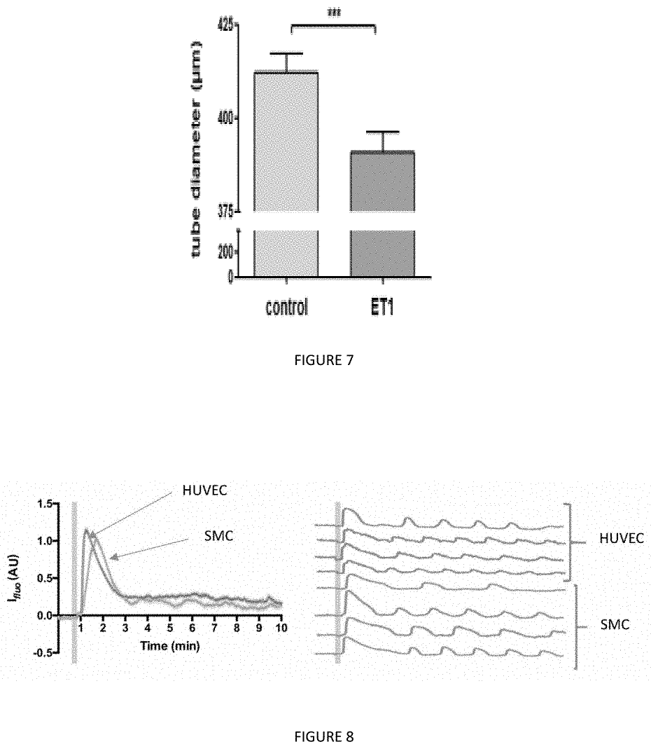

The presence of endothelin 1 causes the contraction of the microfibres, and a significant decrease in the inner diameter, of about 5% (FIG. 7).

Measurements of variations in calcium concentrations, cell type by cell type (human umbilical vein endothelial cells (HUVEC) and smooth muscle cells (SMC)), indicate that under the effect of endothelin 1, a nearly instantaneous increase in intracellular calcium is observed, followed by several oscillations (FIG. 8). This mechanism, classically observed in mature blood vessels, is responsible for triggering and propagating the signal allowing contraction: ET1→↑Ca2+→contraction

These results confirm that the cells that make up the hollow cell microfibres according to the invention behave in the same way as cells of mature blood vessels.

1-16. (canceled)

17. An artificial hollow cell microfibre comprising successively, organized around a lumen:
at least one endothelial cell layer;
at least one smooth muscle cell layer;
an extracellular matrix layer; and optionally
an outer hydrogel layer.

18. The artificial hollow cell microfibre according to claim 17, wherein the outer hydrogel layer is present and comprises alginate.

19. The artificial hollow cell microfibre according to claim 17, wherein the ratio in cm² of endothelial cells to smooth muscle cells in the hollow cell microfibre is between 3:1 and 2:1.

20. The artificial hollow cell microfibre according to claim 17, wherein the endothelial cells are selected from the groip consisting in mammalian umbilical vein endothelial cells (UVEC), dermal microvascular endothelial cells (DMEC), dermal blood endothelial cells (DBEC), dermal lymphatic endothelial cells (DLEC), cardiac mirovascular endothelial cells (CMEC), pulmonary microvascular endothelial cells (PMEC) and uterine microvascular endothelial cells (UtMEC).

21. The artificial hollow cell microfibre according to claim 17, wherein the smooth muscle cells are selected from the group consisting in mammalian vascular smooth muscle cells, lymphatic smooth muscle cells, digestive tract smooth muscle cells, bronchial smooth muscle cells, kidney smooth muscle cells, bladder smooth muscle cells, dermal smooth muscle cells, uterine smooth muscle cells and ciliary smooth muscle cells.

22. The artificial hollow cell microfibre according to claim 17, wherein the endothelial cells have been obtained from induced pluripotent stem (iPS) cells.

23. The artificial hollow cell microfibre according to claim 17, wherein the smooth muscle cells have been obtained from induced pluripotent stem (iPS) cells.

24. The artificial hollow cell microfibre according to claim 17, wherein the inner diameter is between 50 μm and 500 μm. ±10 μm.

25. The artificial hollow cell microfibre according to claim 17, wherein the outer diameter, in the presence of the outer hydrogel layer, is between 250 μm and 5 mm, and the outer diameter in the absence of the hydrogel layer is between 70 μm and 5 mm, ±10 μm.

26. The artificial hollow cell microfibre according to claim 17, said cell microfibre being a blood vessel.

27. The artificial hollow cell microfibre according to claim 17, said cell microfibre being a lymphatic vessel.

28. A process for preparing a hollow cell microfibre, wherein a hydrogel solution and a cell solution comprising endothelial cells and smooth muscle cells in an extracellular matrix are coextruded concentrically in a crosslinking solution capable of crosslinking the hydrogel.

29. The process for preparing a hollow cell microfibre according to claim 28, wherein the cell solution comprises between 20 and 30 vol % cells and between 70 and 80 vol % extracellular matrix.

30. The process for preparing a hollow cell microfibre according to claim 28, wherein the volume ratio of endothelial cells to smooth muscle cells in the cell solution is between 3:1 and 2:1.

31. The process for preparing a hollow cell microfibre according to claim 28, wherein the extrusion rate of the cell solution is between 0.1 and 5 ml/h. ±0.05 ml/h.

32. The process for preparing a hollow cell microfibre according to claim 28, wherein the extrusion rate of the alginate solution is between 1 and 10 ml/h, ±0.5 ml/h.

33. The process for preparing a hollow cell microfibre according to claim 28, wherein an intermediate solution, comprising sorbitol, is coextruded between the alginate solution and the cell solution, the extrusion rate of the intermediate solution being between 0.1 and 5 ml/h, ±0.05 ml/h.

34. The process for preparing a hollow cell microfibre according to claim 28, comprising the additional step consisting in hydrolysing the outer alginate layer after formation of the vessel.
